(19) United States
(12) Patent Application Publication
(10) Pub. No.: US 2004/0120991 A1
(43) Pub. Date: Jun. 24, 2004

(54) Edible films having distinct regions

(75) Inventors: Larry Gardner, Port Murray, NJ (US); Nola Bragg, Hackettstown, NJ (US); Jeannette Dido, Hoboken, NJ (US); Mike Webster, Blairstown, NJ (US); Laura Paluch, Yorba Linda, CA (US); Tiffany Bierer, Fullerton, CA (US)

Correspondence Address: FULBRIGHT & JAWORSKI, LLP, 1301 MCKINNEY, SUITE 5100, HOUSTON, TX, 77010-3095, US

(73) Assignee: MARS INCORPORATED, McLean, VA (US)

(21) Appl. No.: 10/656,509

(22) Filed: Sep. 5, 2003

Related U.S. Application Data
(60) Provisional application No. 60/408,935, filed on Sep. 7, 2002.

(51) Int. Cl. A61K 47/00; A61K 9/70

(52) U.S. Cl. 424/443; 424/439

(57) ABSTRACT

The present invention is related to an edible film having distinct regions, wherein at least one region has a composition that is different from at least one other region.

### CROSS-REFERENCE TO RELATED APPLICATIONS

This application claims priority to U.S. Provisional Application No. 60/408,935 filed Sep. 6, 2002, which is incorporated herein by reference.

### TECHNICAL FIELD

The present invention is related to an edible film having distinct regions, wherein at least one region has a composition that is different from at least one other region.

### BACKGROUND OF THE INVENTION

Edible films providing a single action are known and used in the art. For example, WO 00/18365 discloses a breath freshening film adapted to dissolve in a mouth of a consumer comprised of a water soluble polymer and an essential oil, such as thymol, methyl salicylate, eucalyptol and menthol.

U.S. Pat. No. 4,713,243 discloses a bioadhesive film for delivering therapeutic agents to the oral cavity which is capable of adhering to a wet mucosa surface, composed of a water soluble polymer matrix, a water-insoluble polymer, and a plasticizer. More specifically, the film contains an effective amount of medicament for the treatment of periodontal disease. The film is flexible when wet so as to be unobtrusive to the user after it has been properly positioned and placed in the mouth.

U.S. Pat. No. 5,354,551 discloses a water soluble film pre-segmented into dosage units. The film contains conventional toothpaste ingredients and is formulated with swellable polymers such as gelatin and corn starch as film forming agents which upon application to the oral cavity slowly disintegrate, thereby releasing an active agent incorporated in the film.

U.S. Pat. No. 6,177,096 discloses a film composition containing therapeutic or breath freshening agents for use in the oral cavity prepared from a water soluble polymer such as hydroxypropylmethyl cellulose, hydroxypropylcellulose and a polyalcohol such as glycerol, polyethylene glycol. When applied to the oral cavity, the film exhibits instant wettability followed by rapid dissolution.

Despite the existence in the prior art of rapidly dissolvable oral films, there is not a film that possess more than one distinct region. The films of the present invention provide more than one distinct region. Benefits of having distinct regions are that it provides diversification to enhance consumer appeal or provides diversification to provide different sensations for a consumer in one product. Yet further, the distinct regions of the film of the present invention overcome the need to encapsulate compounds to provide multiple functions. Presently, if a film provided more than one functional compound in the same region, at least one compound would need to be encapsulated to prevent deleterious interactions of the compounds. Encapsulation of compounds delays the effect of the compounds, which is not a desire of the present invention. Since the film quickly dissolves in the mouth, the effect of the compounds needs to be instant, not delayed. Thus, the present invention has used distinct regions to separate compounds, thus preventing compounds from interacting with one another and providing immediate release of the compounds to result in an immediate effect or action in the oral cavity.

Thus, the present invention is the first to provide an edible film having multiple regions, wherein each region can contain a different composition resulting in multiple actions.

### BRIEF SUMMARY OF THE INVENTION

The present invention is drawn to an edible film having at least two separate and distinct regions. The film is rapidly dissolved in the oral cavity.

An embodiment of the present invention is an edible film having at least two distinct regions, wherein at least one region independently comprises a first composition that is different from at least one other region. More specifically, the first composition comprises at least one functional component and/or non-functional component which is different from components in other compositions in other regions of the film. Still further, the film comprises at least one other region having a second composition that is different from the first composition. Thus, each region of the film, which is distinct and/or independent for other regions of the film, contains a composition having at least a functional component and/or non-functional component which is different. The compositions of each of these regions may also contain a variety of other ingredients for example, but not limited to water, sodium, potassium, and/or other additives that are known and used in the art. The regions can be indicated by visual cues, for example, color inclusions, color swirls, or color regions. The film can be administered to a human or companion animal, for example, a horse, cat or dog.

Thus, the film may comprise a region having a first composition having a first functional component. The functional component is selected from the group consisting of hydration agent, refreshment agent, heating agent, comfort agents, breath masking agent, flavor masking agent, tartar reducing agent, plaque reducing agent, pharmaceutical agent and nutraceutical agent. More specifically, the functional component is a hydration agent.

Yet further, the film may comprise an other region having a second composition having a second function component. The first and second functional components are selected from the group consisting of hydration agent, refreshment agent, heating agent, comfort agents, breath masking agent, flavor masking agent, tartar reducing agent, plaque reducing agent, plaque disclosing agent, pharmaceutical agent and nutraceutical agent. More specifically, the first functional component is a hydration agent and the second functional component is a refreshment agent. In preferred embodiments, the hydration agent is an acidulent selected from the group consisting of citric acid, malic acid, succinic acid, adipic acid, tartaric acid, acetic acid, and lactic acid. More preferably, the hydration agent is a combination of citric acid, malic acid and succinic acid. Still further, the refreshment agent is selected from the group consisting of L-menthol, N-ethyl-p-methane-3-carboxamide, N,2,3-trimethyl-2-isopropyl butanamide, monomethyl succinate, Cooler II, Cooler V, Physcool, and Intensate 000379. More preferably, the refreshment agent is Intensate 000379.

Another embodiment of the present invention is a method of preparing an edible film having at least two distinct regions indicated by a color swirl, the method comprising the steps of: mixing at least one water soluble polymer, a first component and a first coloring agent to form a first homogenous mixture; mixing at least one water soluble polymer, a second component and a second coloring agent to form a second homogenous mixture; casting the first homogenous mixture to form a film; drizzling the second homogenous mixture onto the film; smearing the second homogenous mixture with the first homogenous mixture to form a film having at least two distinct regions indicated by the color swirl of the first and second homogenous mixtures; drying the film; and cutting the film into film strips. More specifically, the first and second components are either a functional component or a non-functional component, preferably at least one is functional, more preferably both are functional components.

Another embodiment is a method of preparing an edible film having at least two distinct regions indicated by their different color, the method comprising the steps of: mixing at least one water soluble polymer and a first component to form a first homogenous mixture; mixing a second component and a coloring agent to form a second homogenous mixture; casting the first homogenous mixture to form a film; depositing the second homogenous mixture onto the film; drying the film, wherein the dried film contains at least two regions as indicated by the different colors of the first and second homogenous mixtures; and cutting the film into film strips. In preferred embodiments, the first and second components are a functional component or a non-functional component, preferably at least one is functional, more preferably both are functional components. Preferably, depositing comprises printing or spraying. Depositing comprises words, dots, strips, stars, circles, squares, animal shapes, or food shapes. More preferably, printing is ink jet printing. Still further, a second coloring agent is added to the first homogenous mixture.

Still further, another embodiment is a method of preparing an edible film having at least two distinct regions indicated by color regions, the method comprising the steps of: mixing at least one water soluble polymer and a first component to form a first homogenous mixture; mixing at least one water soluble polymer; a second component and a coloring agent to form a second homogenous mixture; casting the first homogenous mixture to form a film; depositing the second homogenous mixture onto the film; drying the film, wherein the dried film contains at least two regions as indicated by colored regions of the first and second homogenous mixtures; and cutting the film into film strips. In preferred embodiments, the first and second components are a functional component or a non-functional component. Still further, a second coloring agent is added to the first homogenous mixture.

Another embodiment is an edible glitter composition for delivery of at least two components which is rapidly dissolved in the oral cavity, wherein the glitter comprises a first component and a second component. The first and second components are a functional component or a non-functional component. Still further, the edible glitter comprises visual cues, wherein the first component is one color and the second component is a second color.

In preferred embodiments, the functional component is selected from the group consisting of hydration agent, refreshment agent, heating agent, comfort agent, breath masking agent, flavor masking agent, tartar reducing agent, plaque reducing agent, pharmaceutical agent and nutraceutical agent. More preferably, the non-functional component is a flavoring agent or a coloring agent.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION OF THE INVENTION

A. Definitions

As used herein, the use of the word “a” or “an” when used in conjunction with the term “comprising” in the claims and/or the specification can mean “one,” but it is also consistent with the meaning of “one or more,” “at least one,” and “one or more than one.”

As used herein, the term “edible film” or “film” refers to a film that is shaped and sized to be placed in the oral cavity. The film is flexible and adheres to a surface in the mouth, usually the roof of the mouth or the tongue, and quickly dissolves, generally in less than 20 seconds. It is contemplated that the edible films of the present invention can be administered to or consumed by a mammal. Preferred mammals include a human or a companion animal, e.g., horse, dog or cat. Depending upon the mammal or consumer (human or companion animal), one of skill in the art is able to determine the size and which ingredients and/or compositions are suitable and desirable for the consumer. Preferably the film will be sized to fit on the tongue of the consumer and is generally in the range of 1 mm to 30 mm in length, 10 mm to 30 mm in length, and more preferably 15 mm-25 mm in length or any variation therebetween. Yet further, the edible film also refers to glitter or any other film particle.

As used herein, the term “composition” refers to a mixture of components or ingredients that form a distinct region of the edible film. For example, a composition may comprise at least one functional component and a combination of non-functional components to form the mixture. In various embodiments, the composition may contain only non-functional components.

As used herein, the term “functional component” refers to a compound that results in a physiological or psychological effect. It is envisioned that the physiological or psychological effect is a result of the compound as the edible film containing the compound is placed in the oral cavity or is a result of the compound contained in the edible film after absorption into the systemic circulation (bloodstream) of the consumer. Exemplary functional components include, but are not limited to hydration agents, refreshment agents, heating agents, comfort agents, breath masking agents, flavor masking agents, tartar reducing agents, plaque reducing agents, pharmaceutical agents and nutraceutical agents.

As used herein, the term “non-functional component” refers to a compound that does not result in a physiological or psychological effect. Non-functional components can include, but is not limited to selected flavoring agents, coloring agents, film forming agents, plasticizing agents, surfactants, emulsifying agents, stabilizing agents, thickening agents or binding agents. One of skill in the art realizes that selected non-functional components may also be functional, for example selected flavoring agents and/or coloring agents may invoke a given physiological or psychological effect.

As used herein, the term “distinct regions” refers to an area of the edible film that are distinct from each other in composition and preferably visually distinguishable. The size of these regions is preferably greater than 1 mm, 2 mm, 3 mm, 4 mm, or 5 mm in diameter. Each region contains a composition having at least one component that is either a functional component or a non-functional component. It is contemplated that the film of the present invention contains at least two distinct regions where each region contains a composition that is different from the other region. The composition comprises either a functional and/or non-functional component. Still further, it is envisioned that any given region may contain a composition having more than one functional component and non-functional component or it may contain a composition having only non-functional components.

B. Edible Films

The present invention is drawn to edible films having distinct regions. It is envisioned that the films contain at least two distinct regions. Each region contains a composition that is different from the composition in at least one other region of the film. In preferred embodiments, at least one region contains a composition having at least a functional component that results in a physiological and/or psychological effect.

It is contemplated that the film of the present invention contains at least two distinct regions where at least one region comprises a composition that is different from compositions in at least one other region of the film. If the film comprises two distinct regions, then the ratio of the one region to the second region the film may be 50:50. Other ratios that may be preferable depending upon the compositions of the regions include 10:90, 20:80, 30:70, 40:60, 60:40, 70:30, 80:20, and 90:10 or any variation there between. The desired ratio is the ratio of the regions such that an effective amount of the composition having a functional component in the given region is administered to the consumer to achieve the desired effect, such as a physiological and/or psychological effect.

In preferred embodiments, it is envisioned that by providing distinct regions of the edible film, in which a region contains a composition having a functional component, the present invention provides an edible film having regions designed to match receptors in the oral cavity. For example, it is well known by those of skill in the art that the chemoreceptors or “taste buds” are the sense organs that respond to gustatory or taste stimuli. The chemoreceptors are located in the lining of the mouth and throat, however, most of the chemoreceptors are associated with the small elevated projections or papillae on the tongue. There are four primary taste sensations, sweet, sour, bitter and salty. The ability to detect other flavors and tastes is due to the combination of the primary sensations and the also the combination of the sense of smell. Chemoreceptors that are specific for the primary taste sensations are correlated to specific areas of the tongue, for example, sweet is at the tip or middle of the tongue; salty is located on the tip and front sides of the tongue; sour is located on the sides of the tongue; and bitter is located on the back of the tongue. Thus, it is envisioned that by using correlating regions of the film to specific chemoreceptors on the tongue, the present invention can be used to target the primary sensation chemoreceptors to enhance or stimulate the taste sensation.

For example, specific embodiments of targeting the chemoreceptors of the tongue can include a film having a distinct sour region on the sides of the film strip, sweet regions in the center or front of the film strip or bitter regions on the back of the film strip.

One such embodiment of the present invention is an edible film having at least two distinct regions in which at least one region comprises a first composition having a first functional component, a sweet agent, and at least one region comprises a second composition having a second functional component, a sour agent. The combination of sweet/sour components provides the consumer with dual sensations or enhanced sensations. Furthermore, the film can be designed to that the film matches the chemoreceptors on the tongue, which allows the consumer to adjust the intensity of the flavors by adjusting the placement of the film in the oral cavity.

The sweet agent of the present invention can include both natural and artificial sweeteners. Suitable sweeteners include water soluble sweetening agents such as monosaccharides, disaccharides and polysaccharides (e.g., xylose, ribose, glucose (dextrose), mannose, galatose, fructose (levulose), sucrose (sugar), maltose); water soluble artificial sweeteners such as the soluble saccharin salts (e.g., sodium or calcium saccharin salts, cyclamate salts); and dipeptide based sweeteners, such as L-aspartic acid derived sweeteners (e.g., L-aspartyl-L-phenylalaine methyl ester (aspartame)). Preferred sweet agents of the present invention include non-nutritive sweeteners, for example, but not limited to aspartame (NutraSweet, Augusta, Ga.), acesulfame potassium (Nutrinova, Somerset, N.J.) sucralose (Splenda, Splenda Inc. McIntosh, Ala.) and neotame.

Sour agents can include acids or acidulents, for example, but not limited to citric acid, malic acid, succinic acid, adipic acid, tartaric acid, acetic acid, lactic acid, and mixtures thereof.

Yet further, edible films having distinct regions targeted to the primary sensation chemoreceptors of the tongue can also include markers on the film to indicate the direction in which the film should be applied to the tongue or markers to indicate the distinct regions. These markers and/or directions can be applied to the film during standard processing of the edible films.

It is also contemplated that the regions of the inventive edible film can be used to provide enhanced physiological activity in the oral activity. For example, one region in the inventive film can have a greater amount or increased concentration of a functional component, such as salivation agent, to enhance the initial salivation effect and promote mechanical action or movement of the film throughout the oral cavity. In particular, the concentrated functional component may enhance salivation such that the film is rapidly dissolved in the oral cavity.

Yet further, the distinct regions may also prevent and/or minimize deleterious interaction between components. Typically, to prevent interaction between components, at least one component is encapsulated. An encapsulated component prevents interaction with the non-encapsulated component, however, the encapsulated component is delayed in providing its action or effect. Thus, by presenting the components in distinct regions of the inventive edible film, the present invention eliminates the necessity of encapsulating components to prevent deleterious interaction. Yet further, since the components are not encapsulated, the components are instantly exposed in the oral cavity to result in an immediate effect instead of a delayed effect if the components where encapsulated.

Visual cues are also used in the present invention to indicate the distinct regions of the edible film. The visual cues include, color inclusion, color swirl and color printing. The coloring agents are used in amounts effective to produce the desired color. The coloring agents useful in the present invention include pigments such as titanium dioxide, and natural and artificial FD&C approved colors, which may be incorporated in amounts of up to about 5 wt %, and preferably less than about 1 wt %.

C. Film Components

The compositions in the given distinct regions of the film comprise at least one water soluble polymer and other non-functional components. Preferably, the composition also comprises a functional component.

The water soluble polymer used in the films according to the present invention include, but are not limited to pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, tapioca starch, whey protein isolate, casein and mixtures thereof. The concentration of the water soluble polymer used in the film is from about 0.01 to about 99 wt %, preferably about 30 to about 80 wt %, more preferably from about 45 to about 70 wt % and even more preferably from about 60 to about 65 wt %.

1. Functional Components

Functional components result in a physiological or psychological effect. It is envisioned that the physiological or psychological effect is a result of the compound as the edible film containing the compound is placed in the oral cavity or is a result of the compound contained in the edible film after absorption into the systemic circulation of the consumer (bloodstream). Exemplary functional components include, but are not limited to hydration agents, refreshment agents, heating agents, comfort agents, breath masking agents, flavor masking agents, tartar reducing agents, plaque reducing agents, pharmaceutical agents and nutraceutical agents.

In a preferred embodiment of the present invention, the edible film comprises at least two distinct regions, in which one or more regions comprise a composition having a first functional component, which can be a hydration agent, and at least one other region comprises a second composition having a second functional component, which can be a refreshment agent. It is envisioned that this combination of functional components provides an edible film that a consumer can use to alleviate dry mouth. Dry mouth can be caused by a variety of environmental, emotional or physiological factors. Thus, the hydrating/refreshing edible film of the present invention can be used before or after exercise or during hot summer days to hydrate and refresh the oral cavity of the consumer. Yet further, it is contemplated that the hydrating/refreshing edible film can be used to alleviate dry mouth, which is induced by stress, fear, or any other emotional, environmental or physiological factor.

The functional hydration agent of the present invention can contain a salivation agent. For the purposes of the present invention, the term hydration and salivation can be used interchangeably. The salivation agent is present in an amount effective to promote salivation in the oral cavity, thus instant hydration. Any orally acceptable agent that promotes salivation in the oral cavity can be used as a hydration agent. Exemplary hydration agents include acidulents, salts, salt enhancers, monosodium glutamate (MSG), MSG enhancers, flavors and mixtures thereof. Acidulents are preferred hydration agents. Exemplary acidulents include, but are not limited to citric acid, malic acid, succinic acid, adipic acid, tartaric acid, acetic acid, lactic acid and mixtures thereof. In specific embodiments, the hydration agent is a combination of citric acid, malic acid and succinic acid.

As used herein a refreshment agent includes, but is not limited to vanilla, fat, menthol, cooling agents, dextrose and xylitol. Preferred refreshment agents that are used in the present invention are cooling agents. It is envisioned that the cooling agents that are used in the present invention activate the trigeminal nerve. Preferred cooling agents include, but are not limited to L-menthol, N-ethyl-p-methane-3-carboxamide, N,2,3-trimethyl-2-isopropyl butanamide and monomethyl succinate. See e.g., Parrish, M. A., “Market Warms To Physiological Coolants”, Manufacturing Chemist, pp. 31-32 (February 1987). Other exemplary cooling agents for use in the present invention include “COOLER II” and “COOLER V” available from International Flavors and Fragrances, Inc. (IF&F), Dayton, N. J., “PHYSCOOL” available from MANE USA Milford, Ohio and “Intensate 000379” available from Takasago International Cooporation (USA), Rockleigh, N.Y. More preferably, the cooling agent is Intensate 000379. Yet further, other cooling agents include dextrose and xylitol. It is known by those of skill in the art that the breakdown of dextrose and xylitol are endothermic reactions, thus the reaction uses heat to breakdown dextrose and xylitol which results in a cooling effect in the oral cavity. Still further, one of skill in the art realizes any compound that activate trigeminal nerve may be used as cooling agents.

Another preferred embodiment of the present invention is an edible film having at least two distinct regions in which one or more regions comprise a first composition having a first functional component, a heating agent, and at least one other region comprises a second composition having a second functional component, a cooling agent. The combination of hot/cool functional components provides the consumer with dual sensations or enhanced sensations. A preferred heating or hot agent is oleo resin of capsicum or cinnamic aldehyde. Other exemplary heating agents include, but are not limited to hot flavors, which are available from flavor houses, such as S/S 086042 from International Flavors and Fragrances, Inc. (IF&F), Dayton, N.J. The cooling agent can be any cooling agent previously described herein.

Another embodiment of the present invention is an edible film having at least two distinct regions in which one or more regions comprise a first composition having a first functional component, a hydration agent, and at least one other region comprising a second composition having a second functional component, a comfort agent. The combination of the hydration agent and the comfort agent enhances salivation in the oral cavity of the consumer, thus providing hydration and comfort to oral cavity of the consumer. The hydration agent can be any hydration agent previously described herein.

The oral comfort agent is an agent that is present in an amount effective to comfort, e.g., lubricate, coat and/or moisten, the oral cavity. The oral comfort agent may be selected from the group consisting of lipids, proteins, surfactants or mixtures thereof. Preferably the oral comfort ingredient is a lipid. The lipid useful in the present invention may be selected from the group consisting of partially hydrogenated palm kernel oil, medium chain triglycerides, coconut oil, anhydrous milk fat, cocoa butter, corn oil, palm oil, soybean oil, sunflower oil, canola oil and mixtures thereof. In preferred embodiments, tallow lard or chicken fat can be used as an oral comfort agent for companion animals.

In yet another embodiment of the invention, the oral comfort agent may be protein. Proteins may provide comfort to the oral cavity by moisturizing and/or forming a film that can protect and retain moisture. Exemplary proteins include casein, whey, mucins, egg, blood proteins and proteins processed by microparticulation.

Another embodiment of the present invention is an edible film having at least two distinct regions in which one or more regions comprises a first composition having a first functional component, a flavor masking agent, and at least one other region comprising a second composition having a second functional component, a pharmaceutical agent. It is envisioned that the flavor masking agent masks the pharmaceutical to increase palatability of the pharmaceutical. Typically, pharmaceutical ingredients are currently incorporated into tablets, capsules or liquids. Such tablets, capsules and/or liquids are often difficult to administer to a companion animal or human, such as a child or elderly adult. In addition to the difficulty of administering the tablets, capsules and/or liquid, the pharmaceutical ingredient is not platable. Thus, the film of the present invention provides an easy, yet functional delivery system a pharmaceutical agent.

Flavor masking agents are compounds that are designed to inhibit or alter the perception of the undesirable flavor and/or aroma. The flavor masking agent may bind to the compound that results in the undesirable flavor and/or aroma or it may breakdown the undesirable compound into a secondary compound that does not exhibit an undesirable flavor and/or aroma. Exemplary flavor masking agents include anti-bitter compounds (i.e., vanillin and sweeteners) and anti-sweet compounds (gymnema sylvestre).

Yet further, flavor masking agents for companion animals may include flavoring agents that may psychologically affect the animal so that the animal does not taste the “bitter” flavor of the pharmaceutical. These flavoring agents can include, but are not limited to chicken, beef, pork, lamb, beef tallow, chicken broth, beef broth, fish, or other meat products, cheese, and peanut butter.

Examples of pharmaceutical agents that can be used in the present invention include, but are not limited to antimicrobial agents (e.g., triclosan, cetyl pyridium chloride, domiphen bromide, quaternary ammonium salts, zinc compounds, sanguinarine, fluorides, alexidine, octonidine, EDTA); non-steroidal anti-inflammatory drugs (e.g., aspirin, acetaminophen, ibuprofen, ketoprofen, diflunisal, fenoprofen calcium, naproxen, tolmetin sodium, indomethacin); anti-tussives (e.g., benzonatate, caramiphen edisylate, menthol, dextromethorphan hydrobromide, chlophedianol hydrochloride); decongestants (e.g., pseudoephedrine hydrochloride, phenylepherine, phenylpropanolamine, pseudoephedrine sulfate); anti-histamines (e.g., brompheniramine maleate, chlorpheniramine maleate, carbinoxamine maleate, clemastine fumarate, dexchlorpheniramine maleate, diphenhydramine hydrochloride, diphenylpyraline hydrochloride, azatadine meleate, diphenhydramine citrate, doxylamine succinate, promethazine hydrochloride, pyrilamine maleate, tripelennamine citrate, triprolidine hydrochloride, acrivastine, loratadine, brompheniramine, dexbrompheniramine); expectorants (e.g., guaifenesin, ipecac, potassium iodide, terpin hydrate); anti-diarrheals (e.g., loperamide); H₂-antagonists (e.g., famotidine, ranitidine); proton pump inhibitors (e.g., omeprazole, lansoprazole); general nonselective CNS depressants (e.g., aliphatic alcohols, barbiturates); general nonselective CNS stimulants (e.g., caffeine, nicotine, strychnine, picrotoxin, pentylenetetrazol); drugs that selectively modify CNS function (e.g., phenyhydantoin, phenobarbital, primidone, carbamazepine, ethosukimide, methsuximide, phensuximide, trimethadione, diazepam, benzodiazepines, phenacemide, pheneturide, acetazolamide, sulthiame, bromide); antiparkinsonism drugs (e.g., levodopa, amantadine); narcotic-analgesics (e.g., morphine, heroin, hydromorphone, metopon, oxymorphone, levorphanol, codeine, hydrocodone, xycodone, nalorphine, naloxone, naltrexone); analgesic-antipyretics (e.g., salycilates, phenylbutazone, indomethacin, phenacetin); psychopharmacological drugs (e.g., chlorpromazine, methotrimeprazine, haloperidol, clozapine, reserpine, imipramine, tranylcypromine, phenelzine, lithium).

In preferred embodiments, an edible film having a flavor masking agent and a pharmaceutical agent is used to administer pharmaceutical agents to a companion animal. Such pharmaceutical agents that are typically administered to companion animals include, but are not limited to antibacterial agents (e.g., penicillins, cephalosporins and cephamycins, aminoglycosides, quinolones, sulfonamides, tetracyclines, macrolides, lincosamides, chloramphenicaol, bacitracins, vancomycin, rifamycins); antifungal agents (e.g., polyene macrolide antibiotics, imidazoles, flucytosine, griseofulvin, iodides); anthelmintics (e.g., benzimidazoles, imidaothiazoles, tetrahydropyrimidines, macrocyclic lactones); non-steroidal anti-inflammatory agents (e.g., spirin, acetaminophen, ibuprofen, ketoprofen, flunixin, indomethacin, meloxicam, ramifenazone); steroidal anti-inflammatory agents (e.g., hydrocortisone, dexamethasone, betamethasone, deflazacort); chondroprotective agents (e.g., hyaluronic acid, orgotein, copper-contain compounds, glycosaminoglycans); ectoparasiticides (e.g., organochlorines, organophosphates, pyrethrins, avermectins, amitraz, diflubenzuron, cyromazine, methoprene, carbamates, rotenone, phenylpyrazoles); growth promoter agents (e.g., steroid hormones, growth hormone, beta adrenoceptor agonists); anticonvulsant agents (e.g., phenobarbital, diszepam, primidone); tranquilizers/sedative agents (e.g., bensodizepines, butyrophenone, phenothiazines); antipsychotic agents (e.g., acepromazine, haloperidol, fluphenazine, risperidone); mood-stabilizing agents (e.g., lithium, carbamazepine and valprioic acid); antidepressant agents (e.g., tricyclic antidepressants, fluoxetine); analgesic agents (e.g., ketoprofen, naproxen, phenylbutazone); antitussive agents (e.g., morphine, codeine) bet-adrenergic agonists (e.g., epinephrine, albuterol); proton pump inhibitors (e.g., digoxin and digitoxin); vasoactive agents (e.g., hydralazine, chlcium channel blockers, nitroglycerin, nitroprusside); antiarrhythmics (e.g., quinidine, lidocaine, propranolol); appetite stimulators (e.g., diazepam, prednisone) emetic agents (e.g., apomorphine, xylazine); antiemetic agents (e.g., acepromazine, cyclizine, ondansetron); anti-diarrheal agents (e.g., kaolin-pectin, activated charcoal, loperamide); and laxative agents (e.g., castor oil, magnesium sulfate, lactulose).

Yet, another embodiment of the present invention is an edible film having at least two distinct regions in which one or more regions comprise a first composition having a first functional component, a breath masking agent, and at least one other region comprising a second composition having a second functional component, a pharmaceutical agent. It is envisioned that the breath masking agent masks the pharmaceutical to increase palatability of the pharmaceutical as it freshens the breath of the consumer. Thus, the use of the film having a breath masking agent and a pharmaceutical agent provides an easy, yet functional delivery system for a pharmaceutical while providing the consumer with fresh breath.

In specific embodiments, a breath masking agent functions to mask mouth odor or reduces volatile odor causing bacterial sulfur compounds. Breath masking agents that can be used in the present invention include zinc gluconate, citrus oils, fruit essences, peppermint oil, spearmint oil, other mint oils, clove oil, oils of wintergreen anise, menthol, rosemary oil, and parsley seed oils. The pharmaceutical agent can be any of those discussed previously herein.

In specific embodiments, the edible film provides a functional delivery system for parasite control agents to companion animals. For example, pharmaceutical agents such as those effective against heartworms (e.g., diethylcarbamazine, ivermectin or milbemycin oxime), intestinal worms (e.g., piperazine salts, dichlorvos, bebantel, mebendazole, diethylcarbamizine, milbemycin oxime, pyrantel pamoate, oxibendaxole), fleas, and/or ticks (e.g., chlorpyrifos, dichlorvos, malathion, diazinon, lindane, rotenone, permethrin, resmethrin, allethrin, fenvalerate, tetramethrin, zmitrax, fipronil, imidacloprid, lufenuron, dimethyl phthalate benzyl benzoate), could be incorporated into one of the regions of the film as one of the functional components for easy administration to the animal. An exemplary edible film for a companion animal is an edible film having a breath masking agent and heartworm medication. Thus, the animal receives heartworm medication while freshening its breath.

Another embodiment is an edible film having at least two distinct regions in which one or more regions comprise a first composition having a first functional component, a flavor masking agent or a breath masking agent, and at least one other region comprising a second composition having a second functional component, a nutraceutical agent. Similar to the problems encountered with administering pharmaceuticals, nutraceuticals are not easily administered to children, adults or companion animals. It is envisioned that the flavor masking agent or breath masking agent masks the nutraceutical to increase the palatability of the nutraceutical.

Nutraceuticals include herbs, plant extracts, vitamins, minerals, and antioxidants. Exemplary nutraceuticals that can be used in the present invention include, but are not limited to Echinacea purpurea, Echinacea angustifolia, Echinacea pillida, Gingko biloba, saw palmetto, ginseng, cat's claw (uña de gato), cayenne, bilberry, cranberry, grapeseed extract, St. john's wort, cascara sagrada, valerian, elderberry, elder flower, sweet elder, Sambucous nigra, Sambucous canadensis, garlic, Camellia sinensis, Camellia thea, Camellia theifrera, Thea sinensis, Thea bohea, Thea viridis, goldenseal, wild cherry (Rosacea), quercetin, stinging nettles (Urtica), curcumin, bromelain, multiple pancreatic enzymes (protease, protease II, protease III, peptidase, amylase, lipase, cellulase, maltase, lactase, invertase), Emblica officinalis, eicosapentaenoic acid, docosahexaeonic acid, primrose oil, feverfew, ginger root, vitamin E (D-alpha-tocopherol), licorice root (Glycyrrhiza uralensis), aloe vera, horseradish root, L-glutamine, ascorbic acid, antiscorbutic vitamin, rose hips, calcium ascorbate, cevitamic acid, citrus bioflavonoids complex, acerola, zinc or an effective salt thereof, Astragalus membranaceous, Astragalus mongolicus, membranous milk vetch, milk vetch, mongolian milk, dong quai, huangqi, hunag qi, moringa, vitamin A, β-carotene, minerals such as selenium, magnesium, and manganese.

In preferred embodiments, the nutraceuticals for companion animals include, but are not limited to mineral supplements, B vitamins, herbal compounds, plant-based extracts or antioxidants (i.e., provitamin A carotenes (i.e., trans and cis beta-carotenes, all trans and cis alpha-carotenes, and all trans and cis gamma-carotenes), vitamin C, vitamin E, zeta-carotene, trans lycopene, cis lycopenes, phytofluene, phytoene, and tumeric extract (i.e., curcumin), fatty acids and mixtures thereof.

Yet another aspect of the present invention provides an edible film having at least two distinct regions in which one or more regions comprises a first composition having a first functional component, a breath masking agent, and at least one other region comprising a second composition having a second functional component, a plaque disclosing agent. It is envisioned that the breath masking agents function in masking mouth odor or reducing volatile odor causing bacterial sulfur compounds while the plaque disclosing agent identifies dental plaque that is usually transparent and colorless and not easily visible. Thus, the film can be used to identify areas of the mouth where plaque buildup is a problem thereby increasing the awareness of the consumer and probably motivating the consumer to seek early removal of dental plaque.

Plaque disclosing agents that can be used include FD & C Red No. 40, or Allura Red, (the disodium salt of 6-hydroxy-5-[(2-methoxy-5-methyl-4-sulfophenyl)azo]-2 naphthalenesulfonic acid), F D & C Red No. 3 (erythrosine), and D&C Red No. 22 (eocine). Other potential plaque-disclosing agents include FD&C Blue No.1, FD&C Blue No. 2, D&C Green No. 5, and a mixture of FD&C Blue No. 1 and FD&C Yellow No. 5.

Another embodiment of the present invention is an edible film having at least two distinct regions in which one or more regions comprise a first composition having a first functional component, a tarter reducing agent and/or a plaque reducing agent, and at least one other region comprising a second composition having a second functional component, a flavor masking agent or breath masking agent. Thus, the film reduces tarter and plaque, or freshens breath while it reduces tarter.

Dental calculus, or tartar, is recognized as a recurring calcified deposit on the surfaces of the teeth of domestic animals, including dogs and cats, as well as humans. It is generally recognized that dental calculus develops in a sequential process that involves the accumulation of dental plaque and the subsequent calcification of the plaque by saliva, which has very high concentrations of calcium and phosphate. Although calculus, per se, is not directly responsible for the development of oral diseases, it is recognized as a secondary, or contributing, factor in the development of periodontal disease. Once formed, calculus deposits can only be removed through concerted mechanical procedures, i.e., a dental prophylaxis. Thus, the prevention of dental calculus is of importance not only for cosmetic reasons, but also because of dental calculus' secondary role in the development of periodontal disease, and the resultant systemic infections, alveolar bone recession, tooth loss and adverse mouth/breath odors.

Thus, the edible film of the present invention may employ sequestering agents or plaque and tarter reducing agents, which form soluble calcium complexes with the calcium in saliva and dental plaque fluids thereby preventing the usual calcification of dental plaque on the surfaces of teeth. Exemplary agents that are used to sequester or reduce plaque and/or tarter include organic compounds, such as the hydroxycarboxylic acids, including citric acid, ascorbic acid, malic acid, fumaric acid, glutaric acid, acetic acid, tartaric acid, oxalic acid, and the like, and their alkali salts, such as sodium citrate, potassium citrate, etc., as well as their aminopolycarboxylic acid derivatives, for example, ethylenediaminetetraacetic acid. Other tarter and/or plaque reducing agents also include polyphenols (i.e., tea) or sodium hexametaphosphate (HMP) See for example, U.S. Pat. Nos. 5,618,518, 5,296,217, and 5,204,089, which are incorporated herein by reference.

2. Non-Functional Components

In addition, to the functional components, non-functional components such as selected flavoring agents or coloring agents, may be added to the film to impart desirable taste and appearance. Such components may be independently added to the region containing a functional component or to a region that does not contain a functional component. For example, it may be desirable to add a colorant to one region while leaving a second region colorless. Yet further, a coloring agent can be added to both regions. It may also be desirable to employ the same or different flavoring agents to a film having at two regions in that each region contains a different composition having a different functional component or to a film having two regions in that one region contains a composition having a functional component and the second region contains a composition having a non-functional component.

In preferred embodiments, a selected flavoring agent is also added to an edible film having a hydration agent in one region and a refreshment agent in a second region. The flavor agent can be for example orange, citrus or peppermint. In further embodiments, a coloring agent can also be used in combination with the flavoring agent. For example, if the flavoring agent is citrus, then the coloring agent can be Yellow 6 or if the flavoring agent is peppermint, then the coloring agent can be Blue 2.

Other exemplary selected flavoring agents that are used in the present invention include those known to the skilled artisan, such as natural and artificial flavors. These flavorings can be chosen from synthetic flavor oils and flavoring aromatics, and/or oils, oleo resins and extracts derived from plants, leaves, flowers, fruits and so forth, and combinations thereof. Representative flavor oils include, but are not limited to spearmint oil, cinnamon oil, peppermint oil, clove oil, bay oil, thyme oil, cedar leaf oil, oil of nutmeg, oil of sage, and oil of bitter almonds. Also useful are artificial, natural or synthetic flavors such as vanilla, chocolate, coffee, cocoa, citrus oil (e.g., lemon, orange, grape, lime and grapefruit) and fruit essences (e.g., apple, pear, peach, strawberry, raspberry, cherry, plum, pineapple, apricot). These flavorings can be used individually or in admixture. Preferred flavors include mints such as peppermint, artificial vanilla, cinnamon derivatives, and various fruit flavors, whether employed individually or in admixture. Other flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63-258, may be used. Further examples of aldehyde flavorings include, but are not limited to acetaldehyde (apple); benzaldehyde (cherry, almond); cinnamic aldehyde (cinnamon); citral, e.g., alpha citral (lemon, lime); neral, e.g., beta citral (lemon, lime); decanal (orange, lemon); ethyl vanillin (vanilla, cream); heliotropine, e.g., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, e.g., trans-2 (berry fruits); tolyl aldehyde (cherry, almond); veratraldehyde (vanilla); 2,6-dimethyl-5-heptenal, e.g., melonal (melon); 2-6-dimethyloctanal (green fruit); and 2-dodecenal (citrus, mandarin).

In preferred embodiments, selected flavoring agents that are used in an edible film that is being consumed or administered to a companion animal can differ from those that are used for a film that is to be consumed by a human. Exemplary flavoring agents that are typically used for companion animals include, but are not limited to chicken, beef, pork, lamb, beef tallow, chicken broth, beef broth, fish, or other meat products, cheese, and peanut butter.

Exemplary coloring agents include, but are not limited to Colorants can also include natural food colors and dyes suitable for food, drug and cosmetic applications. These colorants are known as FD&C dyes and lakes. The materials acceptable for the foregoing spectrum of use are preferably water-soluble, and include FD&C Blue No. 2, which is the disodium salt of 5,5-indigotindisulfonic acid. Similarly, the dye known as Green No. 3 comprises a triphenylmethane dye and is the monosodium salt of 4-[4-N-ethyl-p-sulfobenzylamino)diphenyl-methylene]-[1-N-ethyl-N-p-sulfonium benzyl)-2,5-cyclo-hexadienimine]. A full recitation of all FD&C and D&C dyes and their corresponding chemical structures may be found in the Kirk-Othmer Encyclopedia of Chemical Technology, Volume 5, Pages 857-884.

The edible film of the present invention can further comprise other non-functional components such as water, additional film forming agents, plasticizing agents, surfactants, emulsifying agents, stabilizing agents, thickening agents and binding agents.

Preferred plasticizing agents include triacetin in amounts ranging from about 0 to about 20 wt %, preferably about 0 to about 2 wt %. Other suitable plasticizing agents include monoacetin and diacetin.

Preferred surfactants include mono and diglycerides of fatty acids and polyoxyethylene sorbitol esters, such as, Atmos 300 and Polysorbate 80. The surfactant can be added in amounts ranging from about 0.5 to about 15 wt %, preferably about 1 to about 5 wt % of the film. Other suitable surfactants include pluronic acid, sodium lauryl sulfate, and the like.

Preferred stabilizing agents include xanthan gum, locust bean gum and carrageenan, in amounts ranging from about 0 to about 10 wt %, preferably about 0.1 to about 2 wt % of the film. Other suitable stabilizing agents include guar gum and the like.

Preferred emulsifying agents include triethanolamine stearate, quaternary ammonium agents, acacia, gelatin, lecithin, bentonite, veegum, monoglyceride, diglyceride, tweens and the like, in amounts ranging from about 0 to about 5 wt %, preferably about 0.01 to about 0.7 wt % of the film.

Preferred thickening agents include methylcellulose, carboxyl methylcellulose, and the like, in amounts ranging from about 0 to about 20 wt %, preferably about 0.01 to about 5 wt %.

Preferred binding agents include starch, in amounts ranging from about 0 to about 10 wt %, preferably about 0.01 to about 2 wt % of the film.

D. Production of an Edible Film having Distinct Regions

For the production of an edible film having distinct regions, it is understood and within the scope of the present invention that the first and second component can be either a functional component and/or a non-functional component as previously described and incorporated herein.

One such method of producing a film having at least two distinct regions is by using glitter as a region. For example, an edible film having at least one region containing a first composition, which has a first functional or non-functional component, is cast into a film. A second composition containing either a functional or non-functional component is cast into a film, dried, and upon drying forms glitter particles. To form at least a second region on the film, the glitter is applied to the film. In further embodiments, the glitter may also contain a second functional component that is different than the functional component contained in the film. Yet further, the glitter may contain a functional component and the film may contain only a non-functional component.

The glitter may contain a coloring agent and the film may be colorless or both the glitter and the film may contain different coloring agents. Yet further, the glitter may be applied to the film in form of strips, stars, circles, squares, letters, words, shapes, etc.

As used herein, the term edible glitter refers to edible particles. Particles of edible glitter may be of any useful size. Preferably, no more than about 5 percent of the particulates will to pass through an ASTM 40 Mesh sieve, and no less than 98 percent of the particulates will be able to pass through an ASTM 4 mesh sieve. More preferably, no more than 15 percent of the edible glitter particles will pass through an ASTM 20 Mesh sieve, and no less than 98 percent of the particulates will be able to pass through an ASTM 4 mesh sieve. The ASTM Mesh size refers to the number of mesh openings per inch. Typically, a 4 Mesh sieve has an open area of 65.9 percent and an opening size of 5,160 μm, a 20 Mesh sieve has an open area of 46.2 percent and an opening size of 860 μm, and a 40 Mesh sieve has an open area of 36 percent and an opening size of 380 μm.

A further embodiment of the present invention is an edible glitter for delivery of at least one functional component which is rapidly dissolved in the oral cavity. The glitter comprises at least one water soluble polymer, a first component and a second component. In preferred embodiments, the first component is a non-functional component and the second component is a non-functional component. More preferably, the first and second components are two different functional components. Visual cues are used to indicate the presence of the two action components. The first component is one color and the second component is a second color. Any of the functional and the non-functional components that are discussed previously herein can be used to produce the edible glitter.

In further embodiments, the present invention provides a method of preparing an edible film having at least two distinct regions indicated by a color swirl. The film is prepared by mixing at least one water soluble polymer, a first component and a first coloring agent to form a first homogenous mixture. Next, at least one water soluble polymer, a second component and a second coloring agent is mixed to form a second homogenous mixture. After all components are mixed, the first homogenous mixture is cast to form a film. Once the film is cast, the second homogenous mixture is drizzled onto the film and smeared with the first homogenous mixture to form a film having color swirl of the first and second homogenous mixtures. After the color swirl is formed, the film is dried and cut into strips. Preferably, the firs and second components are functional.

Another embodiment is a method of preparing an edible film having at least two distinct regions indicated by a color inclusions. First, at least one water soluble polymer, a first component and a first coloring agent is mixed to form a first homogenous mixture. The first homogenous mixture is dried and flaked, braked or cut into particles. Next, at least one water soluble polymer, a second component and a second coloring agent is mixed to form a second homogenous mixture, which is cast to form a film. The particles of the first homogenous mixture are sprinkled onto the film and the film is dried and cut. Preferably, the firs and second components are functional.

Yet further, another embodiment is a method of preparing an edible film having two distinct regions indicated by a color region. First, at least one water soluble polymer, a first component and a first coloring agent is mixed to form a first homogenous mixture. A second component and a second coloring agent are mixed to form a second homogenous mixture. The first homogenous mixture is cast to form a film and the second homogenous mixture is deposited onto the film, which is dried and cut. Depositing can be done by using standard printing, such as ink jet printing and/or spraying techniques that are well known and used in the art. The second homogenous mixture can contain a water soluble polymer. It is envisioned that deposition relates to printing/spraying words, dots, strips, stars, squares, animal shapes, food shapes, or other designs and/or shapes onto the film. Thus, the film has a color base with the first action component and the second action component is deposited onto the film. It is also envisioned that the first homogenous mixture can be colorless and the second homogenous mixture can contain color.

A further embodiment is a method to prepare a striped film. A film forming mixture is prepared having a first functional component. A second film forming mixture is prepared having a second component, which can be functional or non-function. Different coloring agents can be used in the first film forming mixture and the second film forming mixture, or only one coloring agent can be added to either first or the second film forming mixture. Next, the film forming mixtures are cast in stripes at the same time to a desired thickness. These stripes contact each other at the edge forming one film. The striped film is dried and cut.

Casting of the film requires the use of a carrier. The mixture is cast on a releasable carrier and dried. The carrier material must have a surface tension, which allows the film mixture to spread evenly across the intended carrier width without soaking to form a destructive bond between the film and carrier substrates. Examples of suitable carrier materials include glass, stainless steel, Teflon and polyethylene-impregnated paper. Drying of the film may be carried out at high temperature using a drying oven, drying terminal, vacuum drier, or any other suitable drying equipment which does not adversely affect the ingredients of which the film is composed.

After the film is dried, it is segmented into pieces, for example, dosage units, strips, particles, glitter or any other piece that is suitable for delivery of the present invention. Segmenting is performed by die-cutting, slitting-and-die-cutting, laser cutting, or any other technique well known and used in the art. Preferably, the segmented film has a strip width and length corresponding to about the size of a postage stamp, generally about 12 to about 30 millimeter in width and about 20 to about 50 millimeters in length. The film has a thickness ranging from about 15 to about 80 micrometers, and preferably about 30 to 60 micrometers.

### E. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Edible Film

The following table illustrates the ingredients that are combined to produce an edible film having a hydration agent and a refreshing agent.

Hydration

| | | Finished Product |
|---|---|---|
| wet recipe | | Composition |
| 88.70 | aqueous solution of polymers & additives | 58.00 |
| | (approx. 80% water) | |
| 0.00 | Water | 5.00 |
| 3.06 | citric acid | 10.00 |
| 3.06 | malic acid | 10.00 |
| 0.61 | succinic acid | 2.00 |
| 2.75 | sodium citrate | 9.00 |
| 0.08 | acesulfame potassium | 0.25 |
| 0.08 | Sucralose | 0.25 |
| 0.05 | yellow 6 | 0.15 |
| 1.62 | flavorant (citrus) | 5.30 |
| 100.00 | | |

Dry to approximately 5% moisture

Refreshment

| | | Finished Product |
|---|---|---|
| wet recipe | | Composition |
| 92.26 | aqueous solution of polymers & additives | 81.00 |
| | (approx. 80% water) | |
| 4.56 | Water | 5.00 |
| 0.06 | acesulfame potassium | 0.25 |
| 0.06 | Sucralose | 0.25 |
| 1.87 | Takasago cooler 000379 | 8.20 |
| 1.21 | flavorant (citrus) | 5.30 |
| 100.00 | | |

Dry to approximately 5% moisture

Two film mixture solutions are made according to the above recipes. One solution contains the hydration agent and the second solution contains the refreshment agent. The solutions are mixed separately, and then the solutions are cast in a casting box to form distinct regions, such as stripes or other designs. During casting, the solutions can be poured simultaneously to form strips or one solution can be poured and the second can be poured on top to form a design. Once poured, the mixtures remain in distinct regions which are readily distinguishable from the color cues though they flow together to form one strip. The cast film is dried and cut.

### Example 2

### Swirled Edible Film

For an edible film having a color swirl, two film forming mixtures are prepared as described in Example 1. One film mixture contains the hydration agent and the second film mixture contains the refreshment agent. One of the films is cast to a desired thickness. The second base is drizzled onto the first, and is smeared with a blade to achieve a film with a desired pre-drying thickness. The mixtures remain in distinct regions, which are readily distinguishable from the color cues. The film is dried and cut into the desired size and shape.

### Example 3

### Colored Inclusion Edible Film

For a colored inclusion, a film forming mixture containing a hydration agent is prepared as described in Example 1. Next, the film forming mixture is dried and flaked into particles. Flaking the dried mixture into particles requires mechanical manipulation. Next, a second film forming mixture containing the refreshment is prepared as in Example 1. The second film forming mixture is cast to the desired thickness. Then, the particles from the first film forming mixture are sprinkled on the wet film mixture. The film is dried and cut to the desired size and shape.

### Example 4

### Colored Regions on Edible Film

For colored regions, a film forming mixture having a hydration agent is prepared as in Example 1. A colored solution (with or without film base polymers) containing the refreshment agent is prepared. Next, the film forming mixture having the hydration agent is cast to a desired thickness. The, the colored solution is deposited onto the cast film. Depositing may include printing and/or spraying words, dots, stripes, or other shapes.

### Example 5

### Edible Film for Companion Animal

Briefly, water soluble polymers are mixed with a functional component, such as a heartworm medication and a coloring agent to form a first film mixture. Next water soluble polymers are mixed with a second functional component, such as a flavor masking agent and a coloring agent to form a second film mixture. The film mixtures are cast at the same time in a casting box to form distinct regions, such as stripes or other designs. The mixtures remain in distinct regions, which are readily distinguishable from the color cues. The film is dried and cut.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. An edible film having at least two distinct regions, wherein at least one region comprises a first composition that is different from at least one other region.

2. The film of claim 1, wherein the first composition comprises at least a first functional component.

3. The film of claim 2, wherein at least one other region comprises a second composition comprising at least a second functional component.

4. The film of claim 1, wherein at least one region comprises at least a first and a second functional component.

5. The film of claim 1, wherein the regions are indicated by visual cues.

6. The film of claim 5, wherein the visual cues comprise color inclusions, color swirls, or color regions.

7. The film of claim 2, wherein the first functional component is selected from the group consisting of hydration agent, refreshment agent, heating agent, comfort agents, breath masking agent, flavor masking agent, tartar reducing agent, plaque reducing agent, plaque disclosing agent pharmaceutical agent and nutraceutical agent.

8. The film of claim 3, wherein the first and second functional components are selected from the group consisting of hydration agent, refreshment agent, heating agent, comfort agents, breath masking agent, flavor masking agent, tartar reducing agent, plaque reducing agent, plaque disclosing agent, pharmaceutical agent and nutraceutical agent.

9. The film of claim 8, wherein the hydration agent is an acidulent selected from the group consisting of citric acid, malic acid, succinic acid, adipic acid, tartaric acid, acetic acid, and lactic acid.

10. The film of claim 9, wherein the hydration agent is a combination of citric acid, malic acid and succinic acid.

11. The film of claim 8, wherein the refreshment agent is selected from the group consisting of L-menthol, N-ethyl-p-methane-3-carboxamide, N,2,3-trimethyl-2-isopropyl butanamide, monomethyl succinate, Cooler II, Cooler V, Physcool, and Intensate 000379.

12. A method of preparing an edible film having at least two distinct regions indicated by a color swirl, the method comprising the steps of:
mixing at least one water soluble polymer, a first component and a first coloring agent to form a first homogenous mixture;
mixing at least one water soluble polymer, a second component and a second coloring agent to form a second homogenous mixture;
casting the first homogenous mixture to form a film;
drizzling the second homogenous mixture onto the film;
smearing the second homogenous mixture with the first homogenous mixture to form a film having at least two distinct regions indicated by the color swirl of the first and second homogenous mixtures;
drying the film; and
cutting the film into film strips.

13. A method of preparing an edible film having at least two distinct regions indicated by color regions, the method comprising the steps of:
mixing at least one water soluble polymer and a first component to form a first homogenous mixture;
mixing a second component and a coloring agent to form a second homogenous mixture;
casting the first homogenous mixture to form a film;
depositing the second homogenous mixture onto the film;
drying the film, wherein the dried film contains at least two regions as indicated by colored regions of the first and second homogenous mixtures; and
cutting the film into film strips.

14. The method of claim 13, wherein depositing comprises printing or spraying.

15. The method of claim 13, wherein a second coloring agent is added to the first homogenous mixture.

16. The method of claim 13 further comprising mixing at least one water soluble polymer with the second component and coloring agent to form the second homogenous mixture.

17. An edible glitter composition for delivery of at least two components which is rapidly dissolved in the oral cavity, wherein the glitter comprises a first component and a second component.

18. The composition of claim 17, wherein the first and second component are a functional components.

19. The composition of claim 17, wherein the functional component is selected from the group consisting of hydration agent, refreshment agent, heating agent, comfort agent, breath masking agent, flavor masking agent, tartar reducing agent, plaque reducing agent, pharmaceutical agent and nutraceutical agent.

20. The composition of claim 17, wherein a non-functional component is a flavoring agent or a coloring agent.
